(19) United States
(12) Patent Application Publication
(10) Pub. No.: US 2023/0285639 A1
(43) Pub. Date: Sep. 14, 2023

(54) MEDICAL DEVICES HAVING AN IMMEDIATELY DETACHABLE, PERMANENTLY PROLIFERATION-INHIBITING COATING COMPRISING AT LEAST ONE LIMUS SUBSTANCE AND METHOD OF PRODUCTION

(71) Applicant: INNORA GESELLSCHAFT MBH, Berlin (DE)

(72) Inventors: Sebastian SCHURMANN-KAUFELD, Berlin (DE); Nadia BRUNACCI, Berlin (DE)

(73) Assignee: INNORA GESELLSCHAFT MBH, Berlin (DE)

(21) Appl. No.: 18/019,637

(22) PCT Filed: Aug. 5, 2021
(86) PCT No.: PCT/EP2021/071919

§ 371 Date: Feb. 3, 2023

(30) Foreign Application Priority Data
Aug. 5, 2020 (DE) 10 2020 120 717.4

(51) Int. Cl. A61L 29/16; A61L 29/08; A61L 31/08; A61L 31/16

(52) U.S. Cl. CPC A61L 29/16; A61L 29/08; A61L 31/08; A61L 31/16; A61L 2300/802; A61L 2300/216; A61L 2420/02; A61L 2300/416; A61L 2300/63

(57) ABSTRACT

In order to provide medical devices having a coating for local prophylaxis and treatment of undesirable cell proliferation and vasoconstriction, a medical device comprising a coating on at least a portion of the surface is proposed, said coating comprising at least one limus substance in non-encapsulated crystalline form, wherein the at least one non-encapsulated limus substance is applied directly from a solvent mixture of at least one organic solvent and water. The formation of crystals of the limus substance can be brought about or enhanced by slowing down the evaporation of the solvent mixture.

In addition, a method for preparing the medical devices is proposed.

The invention relates to medical devices, comprising a coating on at least a portion of the surface, the coating comprising at least one limus substance in non-encapsulated crystalline form, and to a method for preparing the same.

The hypoperfusion of different tissues is a major cause of functional deficiencies and diseases, mostly in old age, and often also a cause of death. Hypoperfusion is often caused by constriction of the lumina of large blood vessels, the cause of which may in turn be excessive growth of the tissue delimiting the passages, as occurs in the course of healing processes after injury or even surgery.

Uncontrolled and undesirable cell proliferation is a cause of many diseases, including those unrelated to tissue blood supply. Undesirable growth of cells and tissues is known to be able to be influenced by irradiation, by extreme, tissue-damaging temperatures, and with medication. A large number of substances from very different substance classes and with different mechanisms of action are available for inhibiting cell proliferation for the purpose of fighting against tumors. These are usually administered systemically, i.e., by intravenous injection or infusion, or orally. By contrast, the selection of pharmaceuticals for preventing constriction of body passages is almost entirely limited to 2 substance classes: (a) the macrolide antibiotics or limus substances (e.g., sirolimus=rapamycin) and (b) paclitaxel from the class of taxanes. The most prominent effect of these pharmaceuticals is the inhibition of cell proliferation.

Both substances or substance classes are applied locally, i.e. they reach high concentrations in the target area with low systemic exposure. The limus substances are usually applied to coronary stents, slowly released therefrom and transferred to the directly adjacent tissue. Paclitaxel is applied to balloons of balloon catheters and pressed into the adjacent vessel wall when the balloons are momentarily inflated at high pressure. In both coronary arteries and other vessels, paclitaxel prevents vessel wall cells from rapidly proliferating and quickly constricting the vessel lumina. Paclitaxel is also used on stents for peripheral arteries.

The clinical benefit of paclitaxel in reducing vasoconstriction is well recognized. Clinically relevant side effects of paclitaxel after use on balloon catheters have been feared and suspected for years but are not known and have at least not been repeatably proven. Nevertheless, a consensus has developed that the local application of paclitaxel causes a toxic systemic effect and ought to be superseded, if at all possible, by sirolimus or one of the limus derivatives.

This is reflected in Table 1, which has been regularly shown and published in a variety of modifications for a number of years.

TABLE 1
Balloons coated with active ingredient sirolimus,
sirolimus has advantages over paclitaxel

| Property | Sirolimus (or analogues) | Paclitaxel |
|---|---|---|
| Mode of action | Cytostatic | Cytotoxic |
| Margin of safety | 10,000 | 100 |
| Therapeutic range | Wide | Narrow |
| Antirestenotic | Yes - lower late lumen loss | Yes |
| Anti-inflammatory | Yes | No |
| Tissue absorption | Slow | Quick |
| Tissue retention | Short | Long |

Table by Aloke V. Finn, MD, CVPath Institute, Inc, Gaithersburg, MD, USA presented at LINC 2020 (Leipzig Interventional Course, Leipzig, Germany, January 28-31, 2020) (table based on Wessely R, et al. J Am Coll Cardiol. 2006)

However, Table 1 also makes the problem clear: Limus substances are absorbed slowly by tissue when they are applied locally, as intended, and are not retained in tissues as long as paclitaxel. This must be taken as an indication of insufficient efficacy when administered by a balloon inflated only for a short time at the treatment site. The difficulty in developing effective balloon catheters coated with limus substances lies in two facts:
a) To date, practically all published animal testing studies on the inhibition of vasoconstriction by limus-coated balloon catheters have avoided comparison with paclitaxel-coated balloon catheters. One reason may be that limus substances inhibit cell proliferation in pigs to a lesser degree than in humans.
b) Although balloon catheters coated with sirolimus are now commercially available, no study is known to have compared the vasoconstriction inhibition of a limus-coated balloon catheter with that of an uncoated balloon catheter. A clinical trial with a small number of patients compared treatment using a sirolimus-coated balloon catheter with treatment using a paclitaxel-coated balloon catheter (Rosli M A, Abdul Kader MASK, Wan Ahmad W A, Ong T K, Liew H B, Al-Fazir Omar A-F, Zuhdi A S M, Nuruddin A A, Schnorr B, Scheller B. Treatment of coronary drug-eluting stent restenosis by a sirolimus- or paclitaxel-coated balloon JACC: Cardiovasc Intervent, 2019:558-566) DE 10 2013 110 294 A1 discloses applying limus crystals of a mixed size to a balloon surface of a medical device from a polar and a non-polar organic solvent.

The object of the invention is to provide medical devices having a coating for local prophylaxis and treatment of undesirable cell proliferation and vasoconstriction. Said medical devices are to have an efficacy in humans similar to that of paclitaxel-coated medical devices and to offer the advantages of the “limus” substances set out in Table 1.

This object is achieved by medical devices and by a method for preparing said medical devices having the features of the independent claims.

Advantageous embodiments of the inventions are characterized in the dependent claims.

According to the invention, a medical device is provided, comprising a coating on at least a portion of the surface, said coating comprising at least one limus substance in non-encapsulated crystalline form, wherein the at least one crystalline non-encapsulated limus substance is applied directly from a solution together with a water-miscible organic solvent or a mixture of water-miscible organic solvents and water.

The solution does not contain any non-polar solvents since they are not water-miscible.

In addition, according to the invention, a medical device is provided, comprising a coating on at least a portion of the surface, said coating comprising at least one limus substance in non-encapsulated crystalline form, wherein the crystalline non-encapsulated limus substance was applied as a suspension consisting of a water-miscible organic solvent or a mixture of water-miscible organic solvents and water.

The suspension also does not contain a non-polar, water-immiscible organic solvent.

The crystallinity and solution properties of the crystals are determined by the conditions of crystallization and coating.

In the above variants, water and the organic solvent or the individual organic solvents used can be mixed without phase formation.

The medical devices according to the invention are novel and are characterized by the method for preparing the coating.

Preferably, drying of the at least one limus substance applied as a solution or suspension is slowed down or restricted.

Thus, the formation of crystals of the limus substance can be achieved or enhanced by slowing down or restricting the evaporation of the solvent mixture.

The limus substance is a macrolide active ingredient. Sirolimus, everolimus, zotarolimus and biolimus are preferably used.

The medical devices according to the invention can be inserted into the body of a patient and thereby come into temporary or permanent contact with parts of the body.

Preferably, the coating is therefore applied to at least the portion of the surface of the medical device by which the effect is produced by contact with the tissue at the site of treatment after the medical device has been introduced into the human body.

Preferred examples of medical devices are short-term indwelling medical devices, preferably angioplasty balloon catheters and temporary stents, medium-term indwelling medical devices, preferably indwelling catheters, or long-term indwelling implants, preferably stents used to stabilize the lumen of blood vessels or other passages.

The medical devices are preferably coated in a state in which the surface thereof is accessible to the coating to the fullest extent, e.g. stents in a fully expanded state, balloons of balloon catheters in inflated form.

Balloons of balloon catheters folded in a ready-to-use state can be inflated before being coated and can be folded up after being coated, but can also be coated in a loosely or securely folded state.

Alternatively, although not preferred, the medical devices can be coated in the state in which they are introduced into the body, usually in compacted or tightly folded form, possibly already in an introducer catheter or balloons without or also with pre-mounted stents.

In order to achieve a locally effective and locally and systemically tolerable dose of the active ingredients used for the coating, the dose used on the medical device, depending on the product and application, is in a relatively wide range, namely preferably 0.1-20 μg/mm² of the surface of the medical device.

The active-ingredient-containing coatings are commonly applied to the medical devices in a defined dose as a solution or suspension, for example by means of microdosing units, such as microsyringes or pumps, and distributed evenly over the surface.

Insofar as the solvents used contain highly volatile components, contact with a gas phase in metering devices should be restricted to the outlet opening as far as possible.

The common feature of the medical devices according to the invention is the coating with drugs in a dose that is effective on the contacted tissue, said coating being applied as completely as possible to the site of action, at which the coating is rapidly and completely released from the medical device. A portion of the substance released at the site of action forms a reserve that ensures an effect over a long period of time.

The coating preferably comprises one or more antioxidants selected from the following group: ascorbyl palmitate, butylated hydroxyanisole, butylated hydroxytoluene, nordihydroguaiaretic acid, probucol, propyl gallate, resveratrol.

The at least one organic solvent is preferably selected from the following group: methanol, ethanol, isopropanol, tetrahydrofuran and/or acetone.

In addition, the coating can comprise further excipients and/or additives, the proportion of which in the coating may vary and is determined by the intended function.

In addition, a further layer containing further excipients and/or additives can preferably be provided on the coating.

The excipients or the antioxidants used to improve adhesion or delivery of the active ingredients to the tissue are preferably present in proportions of >5 wt % of the limus substance. Antioxidants for chemically stabilizing the limus substances can be used in proportions of 1 wt % or less of the limus substances.

According to a preferred embodiment, the excipients are fatty acid salts or oily substances, such as provitamin B5 (dexpanthenol), in the range of 0.5-50 wt % of the limus substance.

The method according to the invention for coating at least a portion of the surface of a medical device from a suspension containing at least one crystalline limus substance comprises the following steps:

Dissolving at least one limus substance in at least one organic solvent. Adding at least one water-miscible organic solvent, in which the limus substance(s) has a reduced solubility, to the limus solution until the mixture is limus-supersaturated.

This solution or suspension is not used for coating, only for producing limus crystals.

Initiating crystallization of the at least one limus substance to obtain a crystal-containing suspension.

The initiation of crystallization or the crystallization can be achieved by storage at low temperature and/or by ultrasonic exposure of the preparation.

Preferably, before the first crystals form or shortly thereafter, the preparation is cooled and left in the refrigerator (approx. +4° C.) or in the freezer (approx. −20° C.) for 30 min to several days.

Preferably, isolating the crystals of the at least one limus substance from the suspension, and resuspending the crystals in a water-miscible organic solvent or a mixture of water-miscible organic solvents and water, preferably with a proportion of water of 30-50 vol %.

Using the suspension to coat the medical devices. The liquid of the suspension does not contain a water-immiscible organic solvent.

Applying the suspension to the surface of the medical device and drying, with suspended crystals being uniformly distributed and the dissolved portion crystallizing by evaporation of the solvents.

The one preferred variant of the preparation of a coating suspension is set out again in detail below:

To prepare the coating suspension, in a first step, a true solution, for example in ethyl acetate, can be prepared from any (amorphous or crystalline) limus substance or a limus substance mixture, the concentration of the limus substance(s) in the solution preferably being 10 to 300 mg/ml, particularly preferably approx. 100 mg/ml.

An antioxidant, preferably BHT (=butylated hydroxytoluene), can be added to the solution in a concentration of preferably 0.1 to 10 mg/ml.

An organic solvent, such as n-hexane or n-heptane, in which the limus substance is very poorly soluble is slowly added to this solution, while stirring, in a ratio of 1 part limus solution to 1 to 3 parts of, for example, n-hexane, to initiate crystallization of the limus substance(s).

Preferably, before the first crystals form or shortly thereafter, the solution is cooled and preferably left in the refrigerator (approx. +4° C.) or freezer (approx. −20° C.) for 30 min to several days.

The solution can be ultrasonicated to initiate crystallization before cooling or in a pre-cooled state while being ice-cooled and also thereafter to destroy crystal aggregates (not crystals).

The crystals are centrifuged off or filtered and can be dried slowly at room temperature in a stream of air or nitrogen. The use of the wet crystals is possible.

In a second step, water is added to a water-miscible organic solvent or a mixture of organic solvents, each of which is miscible with water, to the extent that the solubility of the limus substance(s) at room temperature is preferably below 30 mg/ml of mixture, more preferably below 10 mg/ml, but always below 50 mg/ml.

The crystals from the first step are added to the solvent mixture in an amount of preferably 10-100 mg/ml.

Alternatively or in a supporting manner, the process of forming or modifying the crystals can occur after the medical device has been coated.

The method according to the invention for coating at least a portion of the surface of a medical device from a solution and generating at least one crystalline limus substance, preferably with slowed-down drying, comprises the following steps:

Dissolving at least one limus substance in at least one water-miscible organic solvent and water, the water content preferably being at least 10%. The at least one organic solvent does not contain a water-immiscible organic solvent.

Applying the solution to the surface of the medical device. Preferably followed by drying, with the drying preferably being slowed down. Preferably, the slowed-down drying occurs by restricting the exchange of air, by a low temperature and/or by a suitable composition of the gas phase (e.g., enrichment with solvent vapor) in the environment of the medical device.

Particularly preferred is the temporary restriction of the evaporation of the solution/suspension liquid(s) by covering the same with a protective cover, which is optionally closed at one end and is made, for example, of Teflon, polypropylene or similar synthetic materials.

According to a preferred embodiment of the method, at least one additional layer of an additive/excipient is applied to the dried layer in a final step.

Furthermore, the solution or suspension can contain an antioxidant, again preferably BHT, in a concentration of preferably 0.01 to 5 mg/ml and any pharmaceutically acceptable additives as stabilizers for the suspension, as chemical stabilizers for the active ingredient(s), as binders for the limus crystals to the surface of the medical devices, as excipients for improving the transfer of the medicinally active substances from the medical device onto or into the tissue.

Such substances are known. Iodinated X-ray contrast media, urea, fatty acids and fatty acid salts, citric acid and citric acid esters, dexpanthenol, and amphiphilic substances, such as lecithin, are preferred.

Suitable excipient concentrations in the coating suspension are 0.1 to 30% (m/v) based on the limus substance in the preparation.

Alternatively or in a supporting manner, the process of forming or modifying the crystals can occur after the medical device has been coated.

A known process is “vapor annealing,” in which the dry drug on the surface of the medical device is exposed to the vapor of a suitable solvent for a relatively long time. Under the influence of the solvent vapor, the desired physical form of the active ingredient is formed.

The process is labor-intensive and time-consuming. Therefore, this conversion of an amorphous (dry) coating to a crystalline coating by subsequent introduction of the coated medical device into a gas phase of a solvent for conversion to crystals (annealing) is not within the scope of the invention.

According to the present invention, as previously described (WO 2011/131 258 A1, WO 2011/131 259 A1, WO 2011/131 678 A1, WO 2014/173 748 A1, WO 2015/039 969 A1), true solutions or suspensions of crystals containing dissolved active-ingredient components can be used for coating. To encourage crystallization of the active ingredients or the growth of the existing crystals, the drying of the liquid on the medical device can be slowed down. This can be achieved, for example, by: selecting only low-volatility solvents; by applying a coating solution portion by portion, the solution being in a diluted form to start with, if necessary, and in a highly concentrated form for subsequent coating passes so as not to redissolve the crystallizate formed in early coating passes; by avoiding air flow; by a particular composition of the surrounding atmosphere; and by a particular temperature of the medical device or the environment. Preferably, a short time after the limus preparation is applied and before the solvent(s) has/have completely dried after coating, the medical device is packaged in a material with limited gas permeability or a container with only a small opening to the environment. A very simple and preferred method is to place a protective cover over the freshly coated balloon that is not yet completely dry. If the balloon was coated in the inflated, non-folded state, the balloon membrane can be folded simultaneously to minimize the balloon diameter.

Significant advantages of the medical devices and coatings described here are: the simple, inexpensive preparation; the possibility of achieving the relatively high amount of active ingredient per mm² of surface area of the medical devices, which is required for limus activity;

stability in storage; the inhibition of lumen constriction and formation of stenoses by neointima proliferation; long-lasting efficacy with regard to the mentioned parameters; and good local tolerability.

Advantageously, in comparison with the prior art, faster and more complete tissue absorption, longer tissue retention and, in particular, a stronger efficacy in animal tests in comparison with previously known limus coatings are achieved.

An in-vitro-measurable prerequisite for a strong and sustained inhibition of the excessive, undesirable neointima proliferation as a result of prolonged tissue retention (see Table 1) is the slow dissolution of the limus substance in aqueous media (see Example 5).

The invention is explained in more detail below using examples:

### EXAMPLE 1

(1) Provision of Sirolimus Crystals

200 mg of sirolimus was dissolved in 2 ml of ethyl acetate; to this solution, 2.5 ml of hexane was added in 0.5 ml portions and mixed. A clear solution was obtained, which was first cooled to −20° C. in the freezer for 10 minutes. Crystallization was initiated by 30 min of ultrasonication, with simultaneous ice cooling.

The sample was stored overnight at −20° C. and then centrifuged at −9° C., and the supernatant was decanted.

The sediment consisting of sirolimus crystals was dried in vacuo.

(2) Coating Preparation

50 mg of the dry crystals thus produced were placed in 1 ml of a solution containing 5 mg/ml of dexpanthenol and 6.5 mg/ml of butylated hydroxytoluene in acetone/water 1:1 (v/v) and mixed. A suspension was obtained.

(3) Coating

Catheter: PTCA, rapid exchange, Creganna, balloon size 3.5×20 mm, inflated, is coated with the suspension.

Sirolimus content/balloon=5.8±0.7 μg/mm²=1469±189 μg

### EXAMPLE 2

(1) Provision of Sirolimus Crystals

500 mg of sirolimus was dissolved in 5 ml of ethyl acetate. To the solution, 6.25 ml of n-hexane was added in 2 ml portions and mixed after each portion. The solution remained clear. To form crystals, the solution was first stored at −20° C. for 10 minutes and ultrasonicated for 30 minutes while being ice-cooled and was then left at −20° C. overnight and centrifuged at −9° C. The supernatant was transferred to another vessel. The sirolimus crystals were dried in vacuo.

(2) Coating Preparations

These crystals were used to prepare the following preparations for coating medical devices:

| Name of | |
|---|---|
| preparation | Composition of the preparations |
| RFlimus-Q-50- | 50 mg/ml of sirolimus + 5 mg/ml |
| DP-5-BHT-0.5 | of dexpanthenol, +0.5 mg/ml |
| | butylated hydroxytoluene in ethanol, water |
| | 60/40 (v/v) |
| RFlimus-S-50- | 50 mg/ml of sirolimus + 5 mg/ml of |
| DP-5-BHT-0.5 | dexpanthenol, +0.5 mg/ml |
| | of BHT |
| | in methanol, water 70/30 (v/v) |

(3) Coating

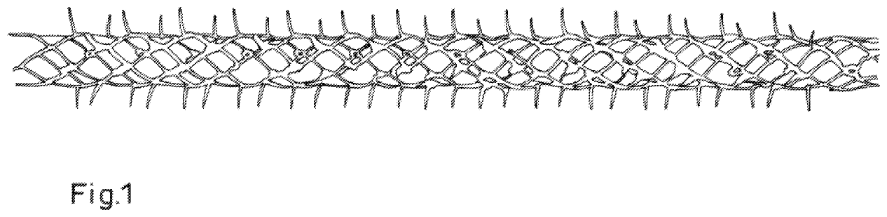

A temporary spur stent system (2^{nd} generation) from ReFlow medical Inc. (San Clemente, USA) was coated

| | | Coating | SIR | SIR |
|---|---|---|---|---|
| Sample | Size | preparation | *(μg) | **(μg/mm²) |
| Coated balloon | 4.0 × 80 mm | RFlimus-Q-50- | 2862 ± 67 | 2.85 ± 0.07 |
| | | DP-5-BHT-0.5 | | |
| Coated spur (stent with | 4.0 × 60 mm | RFlimus-Q-50- | 1297 ± 84 | 8.7 ± 0.6 |
| outwardly directed | | DP-5-BHT-0.5 | | |
| projections, see FIG. 1) | | | | |
| Coated balloon | 4.0 × 80 mm | RFlimus-S-50- | 2693 ± 23 | 2.68 ± 0.02 |
| | | DP-5-BHT-0.5 | | |
| Coated spur (stent with | 4.0 × 60 mm | RFlimus-S-50- | 1396 ± 232 | 9.4 ± 1.6 |
| outwardly directed | | DP-5-BHT-0.5 | | |
| projections, see FIG. 1) | | | | |

*after delivery from the delivery catheter
**cylindrical surface, vessel wall coverage

A spur (stent) coated with RFlimus-Q-50-DP-5-BHT-0.5 is shown in FIG. 1.

### EXAMPLE 3

Comparison of 3 coatings according to the invention with samples of a commercial product (Magic Touch).

TABLE 2
Test sample (SIR = sirolimus, BHT = butylated
hydroxytoluene, THF = tetrahydrofuran):

| | Balloon size | | | | | | |
|---|---|---|---|---|---|---|---|
| | [mm] | Balloon | | | | | |
| | Balloon | surface | | Volume/ | | | SIR |
| | catheter, | area | Composition of the | balloon | Solution/ | SIR | (μg/ |
| Label | type | [mm²] | coating preparation | [μl] | suspension | (μg) | mm²) |
| A2 | 3.5 × 20 | 255 | 40 mg/ml of SIR + | 28 | Solution | 1020 | 4 |
| | Creganna | | 1 mg/ml of Mg | 2 × 14 | | | |
| | | | stearate + | | | | |
| | | | 0.4 mg/ml of BHT | | | | |
| | | | (THF, acetone, | | | | |
| | | | water, acetic acid | | | | |
| | | | 19.5/40/40.05/0.45) | | | | |
| C2 | 3.5 × 20 | 255 | 40 mg/ml of SRL + | 31 | Suspension | 1020 | 4 |
| | Creganna | | 0.4 mg/ml of BHT | | | | |
| | | | (methanol, water | | | | |
| | | | 70/30) | | | | |
| C3 | 3.5 × 20 | 255 | 40 mg/ml of SRL + | 62 | Suspension | 2040 | 8 |
| | Creganna | | 0.4 mg/ml of BHT | | | | |
| | | | (methanol, water | | | | |
| | | | 70/30) | | | | |
| MT | 3.5 × 20 | 255 | ‘Nanolute’ - | ? | Liposomal | ~320 | 1.25 |
| | Magic | | sirolimus-coated | | preparation | | |
| | Touch ™* | | | | ? | | |

*Cortese B, et al, Immediate and short-term performance of a novel sirolimus-coated balloon during complex percutaneous coronary interventions. Cardiovasc Revasc Med (2017), http://dx.doi.org/10.1016/j.carrev.2017.03.025

Preparation of the coating solution/suspensions:

Preparation of the Solution for A2 Coating

A solvent mixture of 3.9 ml of tetrahydrofuran, 8.0 ml of acetone and 90 μl of acetic acid was prepared. In 9.0 ml of the mixture, 6 mg of butylated hydroxytoluene (BHT) was dissolved. 6 mg of magnesium stearate was dissolved in 3.6 ml of BHT solution. To this solution, 2.4 ml of water was added, which was ultrasonicated for 10 minutes. 40 mg of sirolimus was then dissolved in 1 ml of the BHT/magnesium stearate solution.

Preparation of the Suspension for Coating C2 and C3

The sirolimus crystals were provided as described in Example 2.

In 15 ml methanol/water (70/30, v/v), 6 mg of BHT was dissolved (0.4 mg/ml), and 40 mg of SIR/ml was suspended therein.

Coating of A2:

With the folded balloons rotating continuously about the longitudinal axis, 14 μl of the solution was applied twice to the balloons with a period of approximately 30 minutes in between.

Immediately after the coating, wide (inner diameter of 1.3 mm) protective covers were pulled over the balloons.

After 16 hours, the wide protective covers were replaced with narrow protective covers (inner diameter of 1.1 mm).

Coating of C2 and C3:

A protective cover with an inner diameter of approximately 1.2 mm was pushed beyond the uncoated, still folded balloon onto the catheter shaft.

The balloons were inflated immediately before coating.

The suspension was applied to the balloons rotating about the longitudinal axis.

The rotation of the balloons was then continued for 1 minute to allow the coating to dry.

The balloons were then deflated using a vacuum, and the protective covers were pulled back over the coated balloons, with the balloon membranes being folded again.

### EXAMPLE 4

Variations of Coating A2

Preparation of the solution for coating modifications A2a, A2b, A2c, A2d, A2e:

A solvent mixture of 3.9 ml of tetrahydrofuran, 8.0 ml of acetone and 90 μl of acetic acid was prepared. In 9.0 ml of the mixture, 6 mg of butylated hydroxytoluene (BHT) was dissolved. 6 mg of magnesium stearate was dissolved in 3.6 ml of BHT solution. To this solution, 2.4 ml of water was added, which was ultrasonicated for 10 minutes. 40 mg of sirolimus was then dissolved in 1 ml of the BHT/magnesium stearate solution.

Preparation of the solution for coating modifications A3b and A3e

A solvent mixture of 3.9 ml of tetrahydrofuran, 8.0 ml of acetone and 90 μl of acetic acid was prepared. In 10.3 ml of the mixture, 6 mg of butylated hydroxytoluene (BHT) was dissolved. 5.25 mg of magnesium stearate was dissolved in 3.6 ml of BHT solution. To this solution, 2.4 ml of water was added, which was ultrasonicated for 10 minutes. 40 mg of sirolimus was then dissolved in 1 ml of the BHT/magnesium stearate solution.

Modification of the Coating Method:

| Variation | Composition | Coating process |
|---|---|---|
| A2 | 40 mg/ml of SRL + | The folded balloon was coated with a manual Hamilton |
| | 1 mg/ml of Mg stearate + | syringe while being rotated. The coating solution was |
| | 0.4 mg/ml of BHT | divided into two portions. The second portion was |
| | (THF, acetone, water, acetic acid | applied after the first had dried. A wide-lumen |
| | 19.5/40/40.05/0.45) | protective cover was immediately pulled over the still |
| | | wet coating. After the coating had been dried for 12 to |
| | | 24 hours, the wide protective cover was replaced with a |
| | | protective cover with a narrower lumen. |
| A2a | | Coating in 2 portions |
| | | First portion: The inflated balloon was coated with a |
| | | manual Hamilton syringe while being rotated, dried for |
| | | 5 minutes and then folded by applying a vacuum. |
| | | Second portion: Process the same as for A2. |
| A2b | | The balloon was first inflated before coating and then |
| | | folded back again so that the folds were perpendicular |
| | | to the shaft. Otherwise the process is the same as for |
| | | A2. |
| A2c | | The coating was applied with a portion of coating |
| | | solution. The balloon was inflated and coated while |
| | | being rotated. The balloon was then refolded in a |
| | | vacuum, and a wide protective cover was pulled over. |
| | | Otherwise the same as process for A2 |
| A2d | | Process the same as for A2b, but the coating was |
| | | applied with an automatic dispensing system. |
| A2e | | The coating was applied with a portion of coating |
| | | solution. The balloon was first inflated and coated while |
| | | being rotated. After dispensing the first half of the |
| | | coating solution, the pressure was released from the |
| | | balloon while the distribution of the coating solution |
| | | continued. After the coating was finished, a vacuum was |
| | | immediately applied and a wide protective cover was |
| | | pulled over the coating, which was still wet. Otherwise |
| | | the same as process for A2 |
| A3b | 35 mg/ml of SRL + | Process the same as for A2b, but the coating was |
| | 0.875 mg/ml of Mg stearate + | applied with an automatic dispensing system. |
| A3e | 0.35 mg/ml of BHT | Process the same as for A2e |
| | (THF, acetone, water, acetic acid | |
| | 19.5/40/40.05/0.45) | |

The results of the analysis of the modifications are set out in Table 1 under Example 5

### EXAMPLE 5

Differences in the dissolution rate of sirolimus in an aqueous medium

For the composition of the coating preparations, see Table 4 (A1, A2, B1 and D1, columns 2-4; the preparations for C1 and C2 contained crystals according to Example 1, paragraph 1).

Coating:

Before the balloons of groups A1, B1, C1 and D1 were inflated and coated, protective covers with an inner diameter of 1.05 mm were pulled over from the distal region to the proximal region of the balloon shaft. The balloons in question were inflated and coated with the preparations set out in Table 4 with Hamilton microsyringes while being continuously rotated.

After being coated, the balloons were deflated and the protective covers were carefully pulled in the distal direction, with the balloon membranes having been folded back to their original state (before inflation and coating) and the folded balloons being completely covered by the protective covers.

The coating of groups A2 and C2 was carried out as described in Example 3.

Result

Table 4 shows
(a) coating with a suitable dose (≥3 μg/mm², column 6) with all coating variants,
(b) that coating with limus crystals reduces the dissolution rate of the active ingredient (column 7),
(c) that the transfer of the active ingredient onto or into the vessel wall was similar for all preparations (column 8),
(d) that, in the case of the crystalline coating, a much higher drug concentration was found in the arterial tissue 4 weeks after treatment (column 9),
(e) that, in the case of the A coatings, slowed-down drying of the solvents after coating reduces the dissolution rate of the active ingredient in an aqueous medium (column 6).

TABLE 4
Sirolimus (SIR) dissolution test, transfer to/into the vessel wall and indwelling time up to 4 weeks, for the abbreviations see Table 2

| | | | | | | 7 | | | |
|---|---|---|---|---|---|---|---|---|---|
| | 2 | | | | | in-vitro | | | |
| | SIR | | | | | dissolution* | | | |
| | Solution or | | | 5 | 6 | of 1.7 mg | 8 | 9 | 10 |
| | crystal | | | SIR on the | SIR dose | of SIR in | SIR in the vessel wall after treatment** | | |
| | suspension | 3 | 4 | product | by area | 24 hours | Minutes | | 4 weeks |
| 1 | [mg/ml] | Additives | Solvent mixture | [μg] = dose | [μg/mm²] | [%] | % of dose | μg/g | μg/g |
| A1, not | 40/ | 1 mg/ml of | THF, acetone, | 957 ± 33 | 3.8 ± 0.1 | 98 ± 1 | 6.5 ± 4.0 | 187 ± 105 | 0.26 ± 0.07 |
| fractionally | solution | Mg stearate | water, acetic acid; | | | | | | |
| coated, no | | 0.4 mg/ml of | 19.5/40/40.05/0.45 | | | | | | |
| protection | | BHT | (v/v) | | | | | | |
| against | | | | | | | | | |
| evaporation | | | | | | | | | |
| A2, | 40/ | 1 mg/ml of | THF, acetone, | 795 ± 48 | 3.1 ± 0.1 | 17 ± 2 | | | |
| fractionally | solution | Mg stearate | water, acetic acid; | | | | | | |
| coated, with | | 0.4 mg/ml of | 19.5/40/40.05/0.45 | | | | | | |
| protection | | BHT | (v/v) | | | | | | |
| against | | | | | | | | | |
| evaporation, | | | | | | | | | |
| see Example 3 | | | | | | | | | |
| A2a | 40/ | 1 mg/ml of | THF, acetone, | 790 | 3.1 | 29 ± 9 | | | |
| | solution | Mg stearate | water, acetic acid; | | | | | | |
| | | 0.4 mg/ml of | 19.5/40/40.05/0.45 | | | | | | |
| | | BHT | (v/v) | | | | | | |
| A2b | 40/ | 1 mg/ml of | THF, acetone, | 911 ± 37 | 3.6 ± 0.1 | 16 ± 1 | 2.7 ± 0.6 | 97 ± 20 | 24 ± 18 |
| | solution | Mg stearate | water, acetic acid; | | | | | | |
| | | 0.4 mg/ml of | 19.5/40/40.05/0.45 | | | | | | |
| | | BHT | (v/v) | | | | | | |
| A2c | 40/ | 1 mg/ml of | THF, acetone, | 943 | 4 | 90 ± 2 | | | |
| | solution | Mg stearate | water, acetic acid; | | | | | | |
| | | 0.4 mg/ml of | 19.5/40/40.05/0.45 | | | | | | |
| | | BHT | (v/v) | | | | | | |
| A2d | 40/ | 1 mg/ml of | THF, acetone, | 872 ± 157 | 3.4 ± 0.6 | 62 ± 20 | | | |
| | solution | Mg stearate | water, acetic acid; | | | | | | |
| | | 0.4 mg/ml of | 19.5/40/40.05/0.45 | | | | | | |
| | | BHT | (v/v) | | | | | | |
| A2e | 40/ | 1 mg/ml of | THF, acetone, | 774 | 3.1 | 11 | | | |
| | solution | Mg stearate | water, acetic acid; | | | | | | |
| | | 0.4 mg/ml of | 19.5/40/40.05/0.45 | | | | | | |
| | | BHT | (v/v) | | | | | | |
| A3b | 35/ | 0.875 mg/ml of | THF, acetone, | | | | | | |
| | solution | Mg stearate | water, acetic acid; | | | | | | |
| | | 0.35 mg/ml of | 19.5/40/40.05/0.45 | | | | | | |
| | | BHT | (v/v) | | | | | | |
| A3e | 35/ | 0.875 mg/ml of | THF, acetone, | 851 ± 34 | 3.3 ± 0.1 | 9.2 ± 0.5 | 7.2 ± 4.0 | 188 ± 143 | 12 ± 11 |
| | solution | Mg stearate | water, acetic acid; | | | | | | |
| | | 0.35 mg/ml of | 19.5/40/40.05/0.45 | | | | | | |
| | | BHT | (v/v) | | | | | | |
| B1 | 40/ | 0.4 mg/ml of | Acetone, ethanol, | 932 ± 42 | 3.7 ± 0.2 | 97 ± 3 | 5.0 ± 4.2 | 132 ± 87 | 0.45 ± 0.34 |
| | solution | BHT | UV-370; 83/14/3 | | | | | | |
| C1 | 50/ | 5 mg/ml of | Acetone, water | 1469 ± 189 | 5.8 ± 0.7 | 11 ± 0.4 | 2.7 ± 4.2 | 160 ± 127 | 8.6 ± 4.1 |
| | suspension | dexpanthenol | 1/1 (v/v) | | | | | | |
| | | 0.4 mg/ml of | | | | | | | |
| | | BHT | | | | | | | |
| C2 | 40/ | 0.4 mg/ml of | Methanol, water | 878 ± 115 | 3.4 ± 0.5 | 32 ± 2 | 12.6 ± 4.3 | 347 ± 180 | 14 ± 25 |
| | suspension | BHT | 7/3 (v/v) | | | | | | |
| D1 | 30/ | 0.9 mg/ml of | Acetone, water | 1009 ± 14 | 4.0 ± 0.1 | 94 ± 0.4 | 4.8 ± 2.3 | 153 ± 84 | 0.36 ± 0.12 |
| | solution | mannitol, | 75/25 (v/v) | | | | | | |
| | | 0.3 mg/ml of | | | | | | | |
| | | BHT | | | | | | | |

*Coated products in 12 ml of an aqueous 5% (m/v) methyl-β-cyclodextrin (M-β-CD) MβCD solution (5% m/v), measuring the amount of sirolimus dissolved after centrifuging off undissolved components
**For animal testing methodology, see Example 4 and Clever Y P, Peters D, Calisse J, Bettink S, Berg M-C, Sperling C, Stoever M, Cremers B, Kelsch B, Bohm M, Speck U, Scheller B. Novel Sirolimus-coated balloon catheter. In vivo evaluation in a porcine coronary model. Circ Cardiovasc Interv. 2016; 9: e003543. DOI: 10.1161/CIRCINTERVENTIONS.115.003543

### EXAMPLE 6

Preparation and Testing of the Coating C2

For animal testing methodology, see Example 4 and Clever Y P, Peters D, Calisse J, Bettink S, Berg M-C, Sperling C, Stoever M, Cremers B, Kelsch B, Bohm M, Speck U, Scheller B. Novel Sirolimus-coated balloon catheter. In vivo evaluation in a porcine coronary model. Circ Cardiovasc Interv. 2016; 9:e003543.

DOI: 10.1161/CIRCINTERVENTIONS.115.003543

For provision of sirolimus crystals, see Example 2 For coating solution and results, see Table 5 Conclusion
(a) Slowly soluble coating with crystals from an aqueous solvent possible (columns 5, 6, 8, 9, 10).
(b) Slow dissolution of crystalline coating confirmed (column 7).

TABLE 5
Sirolimus (SIR) dissolution test, transfer onto/into the vessel wall
and SIR on balloons after angioplasty, see Table 2 for abbreviations

| | | | | | | | 8 | | |
|---|---|---|---|---|---|---|---|---|---|
| | | | | | | 7 | SIR loss | 9 | |
| | | | | 5 | 6 | in-vitro | on the | Transfer | 10 |
| | | | | SIR on | SIR | dissolution* | simulated | onto/into | Remainder |
| | | | | the | dose by | of 1.7 mg | path to | the vessel | on balloon |
| | 2 | 3 | 4 | balloon | area | of SIR in | the lesion | wall | after use |
| | SIR | Addi- | Solvent | [μg] = | [μg/ | 24 hours | [% of | [% of | [% of |
| 1 | [mg/ml] | tives | mixture | dose | mm²] | [%] | dose] | dose] | dose] |
| C2 a | 40 | 0.5 | Acetone, | 829 | 3.2 | 27 | 15.8 ± | 4.66 ± | 15.7 ± |
| Crystal- | | mg/ml | methanol, | | | | 15.6 | 3.25 | 7.6 |
| line | | of BHT | water v/v: | | | | | | |
| | | | 41/25/34 | | | | | | |

*Coated products in 12 ml of an aqueous 5% (m/v) methyl-β-cyclodextrin (M-β-CD) MβCD solution (5% m/v), measured after centrifuging off undissolved components

### EXAMPLE 7

Effect and Compatibility

Coronary arteries of 20 young domestic pigs were treated with sirolimus-coated catheters according to Examples 3 (A2) and Example 4 (C2). To enhance neointima proliferation caused by overexpansion of the arteries, all balloons were fitted with Coro Large cobalt/chromium stents from Fortimedix, The Netherlands, before sterilization. Immediately after treatment, the luminal diameter of the slightly overexpanded coronary vessel segments was measured, and the measurement was repeated after 4 weeks. The reduction in lumen diameter during the 4 weeks is termed “late lumen loss” (LLL) and characterizes the undesirable constriction of the vessels by neointima proliferation. The desired inhibition of neointima proliferation and thus the inhibition of the constriction of the treated arterial segments results, in part, from the difference in LLL between arteries treated with uncoated balloons and arteries treated with the sirolimus-coated balloons. The results can be found in Table 6.

TABLE 5
Coating solutions and volumes/balloon, see Table 2 for abbreviations

| | Name of | Composition of | Volume/ | |
|---|---|---|---|---|
| | coating | the coating | balloon | Solution/ |
| Group | solution | solutions | [μl] | suspension |
| A2 | Aco-SRL-A2 | 40 mg/ml of SIR + | 2 × 14 | Solution |
| | | 1 mg/ml of Mg stearate + | | |
| | | 0.4 mg/ml of BHT | | |
| | | (THF, acetone, water, acetic | | |
| | | acid 19.5/40/40.05/0.45, v/v) | | |
| C2 | Aco-SRL-C3 | 40 mg/ml of SIR + | 31 | Suspension |
| | | 0.4 mg/ml of BHT | | |
| | | (methanol, water 70/30) | | |

TABLE 6
Inhibition of neointima proliferation/constriction
of coronary artery lumen after angioplasty in pigs
A2, C2 balloon coatings according to Table 5, U = uncoated

| | Balloon catheter/coating | | |
|---|---|---|---|
| | A2 | C2 | U |
| | Number of balloon catheters/arteries | | |
| | n = 12 | n = 12 | n = 12 |
| Sirolimus content [μg/mm²] | 3.1 ± 0.2 | 3.5 ± 0.4 | 0 |

Quantitative coronary analysis (angiograms)

| | | | |
|---|---|---|---|
| Minimum arterial lumen diameter, | 2.22 ± 0.24 | 2.28 ± 0.28 | 2.21 ± 0.31 |
| segment before treatment [mm] | | | |
| Overexpansion due to stent implantation, | 1.39 ± 0.12 | 1.27 ± 0.13 | 1.31 ± 0.13 |
| ratio | | | |
| Minimum arterial lumen diameter 4 weeks | 2.38 ± 0.24* | 2.46 ± 0.23* | 1.74 ± 0.57 |
| after treatment [mm] | | | |
| LLL [mm] | 0.67 ± 0.26* | 0.60 ± 0.28* | 1.14 ± 0.55 |
| In-stent lumen diameter | 21.6 ± 6.81* | 16.62 ± 8.90* | 39.7 ± 19.0 |
| Stenosis in % of diameter after treatment | | | |
| and stent implantation | | | |

Histomorphometry (microscopy) of arteries
extracted 4 weeks after treatment

| | | | |
|---|---|---|---|
| Lumen, cross-sectional area (mm²) | 4.91 ± 0.77* | 4.73 ± 0.89* | 3.11 ± 1.48 |
| Lumen loss (cross-sectional area) | 27.3 ± 8.8* | 25.3 ± 8.0* | 54.8 ± 18.1 |
| % of area after stent implantation | | | |
| Lumen loss (diameter) | 14 | 13 | 33 |
| % of the diameter of the stent | | | |
| Neointima area [mm²] | 1.85 ± 0.71* | 1.60 ± 0.54* | 3.54 ± 0.97 |
| Stenosis area [%] | 13.9 ± 4.5* | 12.9 ± 4.0* | 33.4 ± 14.4 |

*p < 0.01 versus uncoated

The animals did not show any clinically apparent signs of intolerance either acutely or during the 4-week observation period. ECG, blood pressure, angiography and histology confirmed the high level of tolerance of the coatings.

1-15. (canceled)

16. A medical device, comprising a coating on at least a portion of the surface thereof, wherein the coating comprises at least one limus substance in non-encapsulated crystalline form, and wherein the at least one crystalline non-encapsulated limus substance:
i) was applied directly from a solution containing a water-miscible organic solvent or a mixture of water-miscible organic solvents and water; or
ii) was applied as a suspension consisting of a water-miscible organic solvent mixture or a mixture of water-miscible organic solvents and water,
optionally, drying of the at least one limus substance applied as a solution according to (i) or suspension according to (ii) was slowed down or restricted,
and wherein, further, the organic solvent is selected from a group consisting of methanol, ethanol, isopropanol, tetrahydrofuran, acetic acid and/or acetone.

17. The medical device according to claim 16, characterized in that the optional drying was carried out, and said drying was performed by pulling over a pipe or tube.

18. The medical device according to claim 16, characterized in that the coating further comprises one or more antioxidants selected from the following group: ascorbyl palmitate, butylated hydroxyanisole, butylated hydroxytoluene, nordihydroguaiaretic acid, probucol, propyl gallate, and resveratrol.

19. The medical device according to claim 16, characterized in that the coating further comprises one or more excipients and/or additives.

20. The medical device according to claim 19, characterized in that the excipients are antioxidants in proportions of >5 wt % of the limus substance.

21. The medical device according to claim 19, characterized in that the excipients are fatty acid salts in the range of 0.5-50 wt % of the limus substance.

22. The medical device according to claim 16, characterized in that a further layer comprising one or more excipients and/or additives is applied.

23. The medical device according to claim 22, characterized in that the excipients are antioxidants in proportions of >5 wt % of the limus substance.

24. The medical device according to claim 22, characterized in that the excipients are fatty acid salts in the range of 0.5-50 wt % of the limus substance.

25. The medical device according to claim 16, characterized in that the medical device is an angioplasty balloon catheter, an indwelling catheter, or a stent.

26. The medical device according to claim 16, wherein said at least one limus substance is selected from Sirolimus, everolimus, zotarolimus, and biolimus.

27. A method for coating at least a portion of the surface of a medical device with at least one crystalline limus substance, said comprising:
a) dissolving or suspending at least one limus substance in at least one water-miscible organic solvent and water to form a supersaturated solution or to maintain true solubility, followed by either:
b1) applying the supersaturated solution or the true solution to the surface of the medical device, and crystallizing; or
b2) applying the suspension to the balloon surface; and
c) drying.

28. The method according to claim 27, characterized in that the drying according to c) is slowed down.

29. The method according to claim 28, characterized in that the drying c) is slowed down by placing the medical device in a pipe or tube, by restricting air exchange, by a low temperature and/or by the composition of the gas phase in the environment of the medical device.

30. The method according to claim 27, characterized in that, in a final step d), at least one additional layer of an additive/excipient is applied.

31. The method according to claim 27, characterized in that, in addition to the at least one limus substance, one or more antioxidants are applied.

32. The method according to claim 31, wherein said one or more antioxidants is/are selected: ascorbyl palmitate, butylated hydroxyanisole, butylated hydroxytoluene, nordihydroguaiaretic acid, probucol, propyl gallate, and/or resveratrol.

33. The method according to claim 27, wherein said at least one limus substance is selected from Sirolimus, everolimus, zotarolimus, and biolimus.
